(19) United States
(12) Patent Application Publication
(10) Pub. No.: US 2010/0247570 A1
(43) Pub. Date: Sep. 30, 2010

(54) High Mannose Glycoprotein Epitopes

(75) Inventors: Yvonne Rosenberg, Washington, DC (US); Markus Sack, Aachen (DE); Rainer Fischer, Monschau (DE)

Correspondence Address: MDIP LLC, POST OFFICE BOX 2630, MONTGOMERY VILLAGE, MD, 20886-2630, US

(73) Assignee: PROCELL CORPORATION, Rockville, MD (US)

(21) Appl. No.: 12/514,312

(22) PCT Filed: Nov. 13, 2007
(86) PCT No.: PCT/US2007/084486

§ 371 Date: May 28, 2010

Related U.S. Application Data
(60) Provisional application No. 60/865,507, filed on Nov. 13, 2006.

(51) Int. Cl. A61K 39/21; A61P 31/12; A61P 31/18; C12N 15/09

(52) U.S. Cl. 424/208.1; 435/69.3

(57) ABSTRACT

Glycoproteins containing high mannose oligosaccharides are obtained and used to generate antibody to the mannose portion of the molecule. Such antibodies can be used in a diagnostic assay. The glycoproteins can be used to generate neutralizing antibody.

### FIELD OF INVENTION

The invention relates to glycoproteins with high mannose epitopes that are immunogenic in mammals, which can be used, for example, as vaccines. In particular, the invention provides a means for making and using glycoproteins expressing high mannose oligosaccharides. The immunogenic high mannose glycoproteins of interest comprise immunoprotective epitopes and thus elicit production of protective (neutralizing) antibodies that recognize the high mannose epitopes on pathogens, such as viruses, such as HIV.

### BACKGROUND

Many pathogens exhibit a diverse array of glycosylated (glyco) proteins (Kuo et al., J Clin Invest 98:2813-2818, 1996). Many viruses, in particular, have heavily glycosylated surface envelope glycoproteins, e.g., West Nile virus and HIV. Certain of those glycans or oligosaccharides (sugars) of and on the pathogen proteins can be of three types, high mannose, hybrid or complex (U.S. Pat. No. 7,053,067) and can attach to receptors on cells, e.g., CD209 and the mannose-6-phosphate/insulin-like growth factor 2 receptor (Puolakkainen et al., Infec Imm 73:4620-4625, 2005), resulting in infection of the cells by the pathogen. In some viral infections which can use different receptors for entry into a cell, high mannose glycans can act as immunogenic conformational epitopes capable of inducing neutralizing antibodies.

The 2006 AIDS Epidemic Update reported 2.9 million AIDS deaths in 2006, with 4.3 million new infections and 40 million HIV⁺ individuals worldwide at year's end. That dramatically highlights the need for an efficacious HIV vaccine, particularly for those developing countries where the majority of people remain untreated and/or untested. Many different prophylactic and therapeutic prime-boost vaccine strategies using combinations of plasmid DNA, live recombinant viral vectors (e.g., canary pox, vaccinia and adenovirus) and protein immunizations, have been tested in humans and macaques in attempts to delineate the most effective anti-viral immune responses. To date none of those candidate have been successful, including a recently terminated Merck adenovirus-based vaccine (carrying HIV gag, pol and nef genes) trial. (1-6).

The component of the adaptive immune system considered in recent years to be important for controlling HIV/SHIV/SIV replication and disease progression is the differentiation and expansion of virus-specific CD8 cytotoxic (CTL) cells (7-9). The inverse relationship between CD8 levels and SIV viral load is evident following CD8 depletion of acutely and chronically infected macaques (10,11). In addition, decline in primary viremia and the setpoint viral loads seen in HIV-infected and SIV-infected individuals, as well as vaccinated macaques, have been thought to correlate closely with the cytotoxic T cell (CTL) levels as opposed to neutralizing antibody (NA) induction (12-14).

On the other hand, protection in HIV HEPS (highly exposed persistently seronegative) survivors does not correlate well with levels of anti-HIV CTL and in addition, recent studies characterizing escape mutants have unfortunately indicated the fragility of the seemingly robust anti-viral CD8⁺ cell responses in terms of controlling replication. Such viral escape from CTL now appears to be a hallmark of acute SIV infection; CTL with high functional avidity rapidly selecting for those escape mutants in which the CTL epitope either fails to bind to the cognate MHC class I molecules or forms a complex with MHC that is not recognized by the T-cell receptor (15). The limitations that escape mutants impose on long term protection in vaccinated individuals underscores the need for broader vaccine-induced immunity, either by the generation of additional CTL clones to epitopes in HIV Nef, Pol and Rev proteins, for example, that weakly select for escape mutants long term and/or the induction of strongly neutralizing antibody to determinants on surface gp120 and transmembrane gp41 molecules.

Recombinant HIV envelope (env) vaccines have historically been disappointingly poor at eliciting antibodies that neutralize primary isolates despite employing many approaches (16). Nevertheless, recent findings continue to support the idea that neutralizing antibody plays an important role in acute infections and in preventing disease progression, e.g., several broadly crossreactive human neutralizing monoclonal antibodies (mAbs) have been generated e.g., IgG1b12 (a CD4 binding site mAb), 2F5 and 4E10 (membrane proximal external region (MPER) binding mAbs), and the gp120 glycan binding, 2G12 (17,18). 2G12 is the only known antibody which binds solely to a carbohydrate epitope of HIV. In combination, those antibodies have been shown to passively protect macaques against vaginal transmission with pathogenic SHIV (19,20).

A dramatic reduction in 2G12 binding to gp120 was observed following cleavage of specific mannose linkages by e.g., Endo H treatment, which leaves only the core asparagine-linked GlcNAc; Jack Bean mannosidase treatment which leaves Man1GlcNAc2; and A. saitoi mannosidase treatment, which removes a single mannose from the D2 and D3 arms and two mannoses from the D1 arm of Man9 GlcNAc2, leaving Man5 GlcNAc2 (21). Similar to the inhibition of 2G12 binding with monomeric mannose, 2G12 binding to gp120 is also inhibited by the cyanobacterial protein, cyanovirin, (CVN), which is also known to inhibit HIV-1 entry into target cells. This protein binds specifically to the Manα1→2Man termini of Man8 GlcNAc2 and Man9 GlcNAc2 (21,22). Thus, the 2G12 epitope appears to be comprised of a cluster of high mannose residues contributed by up to three different oligomannose chains (probably Man 8 or Man 9) on the outer face of gp120 without any involvement of protein side chains.

Studies from several laboratories (21-23) revealed, (i) site-specific analysis of the N-linked glycosylation of gp120 initially showed an assortment of oligomannose glycoforms attached at 5 asparagine residues (N295, N332, N339, N386 and N392) which lie on either side of the V3 and V4 loops of the outer face of gp120; (ii) a comparison of the primary sequence of 18 isolates that are neutralized by 2G12 indicated that the N-linked carbohydrate signal sequences associated with N295, N332 and N392 are highly conserved among the members of the panel (with only one exception at each position); (iii) extensive sequencing of pseudotyped viruses carrying acute/early Env, all of which are neutralized by 2G12, indicated that two clones which lacked the predicted N-linked glycosylation sites at residues 339 and 386 remained sensitive to 2G12, confirming that those two residues are not always essential to 2G12 recognition (23); (iv) exoglycosidase and endoglycosidase digestion of the oligomannose glycans of monomeric gp120 provided the critical nature of the Manaα1→2Man structure; and (v) more recently, Dacheux et al. (24) confirmed that the glycosylation sites, 295, 332 and 392, are essential for 2G12 binding and, three of four well characterized HIV sequences on certain viral isolates during primary infection did not expose the 2G12 neutralising epitope in contrast to isolates that expressed 2G12 four years later (24). In that study, the early HIV env glycoprotein did not possess the critical glycosylation sites required to generate the 2G12 epitope.

Recreating the carbohydrate epitope of 2G12 has not been fruitful. 2G12 antibody is not commonly detected in the sera of HIV⁺ patients. Thus, purely carbohydrate epitopes are not the focus of the HIV vaccine community.

In addition to the 2G12 carbohydrate epitope, another seemingly glycan-dependent HIV epitope in the V2 domain of HIV-1 gp120 is recognised by a neutralizing chimpanzee mAb (30). However, that mAb binds to both carbohydrate and polypeptide sites. The carbohydrate may be complex.

N-linked glycosylation producing high mannose proteins occurs in the endoplasmic reticulum (ER). In the case of HIV env, when the mannosylated proteins exit from the ER and “mature” along the secretary pathway through the Golgi, there is a trimming of high mannose glycans and the formation of complex or hybrid glycans resulting in potential alteration or loss of the high mannose structures that were present in the ER. A glycan analysis of plantibodies (antibodies made in plants) and secreted HIV gp120 produced in CHO cells (25) revealed that the percentage of N-linked glycans found on ER-retained proteins (90-100% high mannose) was higher when compared with secreted proteins (˜50-60% high mannose). A clipping of high mannose glycans, and further glycan modification leads to N-glycan heterogeneity in mature or secreted molecules, with high mannose structures being expressed at low levels or, possibly on any one glycoprotein, not at all. Moreover, the complex and hybrid mannose structures may dampen a response to any high mannose structures present. Also, a cluster consisting of one or two high mannose glycans and one complex glycan might still be recognized by a specific antibody, but the reduced affinity would mean that the stimulation signals generated could be insufficient to cause activation and differentiation of unprimed virgin B cells.

Some neutralizing epitopes, e.g., b12 and 2F5, are known to be present on acute/early HIV Env proteins (24), although it is unclear why neutralizing antibodies are not commonly made against these epitopes in HIV infected or vaccinated individuals. Others, like the 2G12 epitope, may not be present early because the N-linked glycosylation sites required to create the 2G12 epitope may not be present in primary undiversified viruses (24) or are unexposed. Many other possibilities for making a neutralizing vaccine have been proposed, but to date, none have met with success.

The poor sensitivity of primary isolates to neutralization, as compared to lab adapted strains, is believed to be due to tertiary folds on the gp120 core, the positioning of N-linked glycans or to possible subunit interactions leading to inaccessibility of antibody binding sites in the context of the native glycoprotein complex on the virion (26-28). A proposed concept suggested that molecular mimicry may exist between certain neutralizing epitopes on HIV envelope and normal host antigens resulting in tolerance (29).

The development of plants expressing foreign proteins has recently emerged as a highly promising approach for inexpensively producing biologically active proteins. To date, genetically engineered plants have been used to produce proteins as diverse as sIgA, vaccine antigens, enzymes and growth factors (31-36). Also, plant viral proteins assembled as viral particles have been used as carrier proteins in animal vaccine models. In terms of HIV, the conserved gp41 neutralizing epitope (2F5), when displayed as part of the coat protein (CP) of potato virus x, was found to be an efficient inducer of neutralizing antibody in i.n. or i.p. immunized normal and hu-PBL-SCID mice (34). Similarly, CP of alpha mosaic virus successfully served as a carrier molecule for antigenic HIV (gp120 V3) and rabies peptide epitopes for the induction of neutralizing antibodies (35).

Plant-derived proteins offers several advantages (31): (i) production of a large amount of biomass without a need for sophisticated facilities; (ii) safety, due to a lack of contamination with animal/human pathogens when compared to recombinant microorganisms, human fluids, animal cell lines or transgenic animals; and (iii) the relative ease of genetic manipulation.

Recombinant proteins produced by plants are essentially indistinguishable from the wild type mammalian protein in many respects. Protein synthesis, secretion and chaperon-assisted protein folding together with the post-translational modification processes, such as signal peptide cleavage, disulfide bond formation and initial glycosylation, are very similar between plant and animal cells. Plants offer another important advantage in that the plant expression system provides a model for controlling subcellular trafficking and targeting of protein synthesis to different cell compartments (25) to achieve differential glycosylation.

### SUMMARY

The instant invention relates to making and using recombinant glycoproteins expressing an immunogenic high mannose epitope. Such high mannose glycoproteins, which can be found on a pathogen, can be used as new targets for protective antibodies to a high mannose epitope. Such glycoproteins can find use when the cognate epitope is not highly expressed on a pathogen, and hence, generally is non-immunogenic in a particular host, or may be expressed at different stages during the course of an infection. The high mannose-specific antibodies arising from such immunogens also can find use as a passive vaccine or can be used in a variety of diagnostic assays.

Those and other goals have been achieved by the invention taught herein, including the manipulation and production of recombinant glycoproteins in, for example, plants, to express high mannose forms of pathogen glycoproteins.

Additional features and advantages are described herein, and will be apparent from the following Detailed Description of the Invention.

### DETAILED DESCRIPTION OF THE INVENTION

Glycoproteins express immunogenic epitopes composed of peptides, glycans (sugars) or mixtures of peptides and sugars (e.g. glycan-dependent peptidic epitopes). Glycosylation can be N-linked or O-linked, the former occurring when the carbohydrate is added to asparagines residues, and the latter occurring when the carbohydrate is added to either a serine or a threonine residue. Glycosylation can be variable, with sugars occurring in a single chain or being simply branched containing, for example, two, three or four chains (biantennary, triantennary or tetraantennary) or branched in heterogeneous and complex ways.

The instant invention arises from the heretofore unappreciated finding that the intracellular processing of glycoprotein in the ER can be disrupted to produce glycoprotein enriched for high mannose epitopes. Retention motifs, such as KDEL, have been used to study trafficking, folding, degradation and quality control of proteins (Heleius & Aebi, Ann Rev Biochem 73, 1019-1049, 2004; and Ruddock & Molinari, J Cell Sci 119 (Pt. 21), 4373-4380, 2006).

It was now recognized that by disrupting, interrupting, waylaying etc. the movement of a nascent glycoprotein from the endoplasmic reticulum to the Golgi, maturation of the glycoprotein is terminated, resulting in a polypeptide expressing a number of mannose residues, many as high mannose structures, many having a mannose as the terminal sugar residue on a chain. Those intracellular transient glycoprotein intermediates are collected and used as high mannose immunogens in the practice of the instant invention to detect and to react with cognate antigens that heretofore were not believed to be expressed by pathogens, perhaps due to pathogen diversity, or were not effectively expressed by a pathogen to stimulate antibody production. The epitopes of interest can be carbohydrate only, that is, directed to mannose only, or to a combination of a high mannose and a polypeptide.

Thus, high mannose forms of HIV env glycoprotein, synthesized and retained in the plant endoplasmic reticulum (ER) by KDEL tagging and targeting has been expressed and demonstrated to have both strong binding to 2G12 antibody, as well as a very slow binding dissociation rate as revealed by a BIAcore device (Piscataway, N.J.).

While the instant invention finds particular use with N-linked glycoproteins, other methods for making high mannose glycoproteins are taught herein, and can be practiced with O-linked glycoproteins as well.

For the purposes of the instant invention, “high mannose” relates to a carbohydrate structure composed of at least 4 residues, at least 5 mannose residues, at least 6 residues, at least 7 residues, at least 8 residues, at least 9 residues, at least 10 mannose residues and so on. The carbohydrate structure can be branched to contain two or more branches. Generally, a mannose residue is the terminal sugar of each branch.

Also, “immunogen” and “antigen” are used interchangeably herein as a molecule that elicits a specific immune response containing an antibody that binds to that molecule. That molecule can contain one or more sites to which a specific antibody binds. As known in the art, such sites are known as epitopes. In the instant invention, such epitopes are those on or including high mannose carbohydrate structures displayed on glycoproteins. Some epitopes may be a combination of a high mannose structure and a polypeptide portion of the glycoprotein.

The high mannose glycoproteins of interest can be made, for example, recombinantly, enzymatically, metabolically, synthetically or a combination thereof. Thus, a suitable host cell can be treated, exposed and the like to agents other than retention motifs which suppress movement of a nascent glycoprotein to the Golgi. Because the proteins of interest are associated with the ER, a suitable means for obtaining the high mannose glycoproteins of interest is to lyse the cells and to isolate the glycoprotein of interest from the lysate. That purification can occur, for example, using an affinity chromatography means, using, for example, an antibody directed to the protein portion of the glycoprotein molecule, as known in the art.

The recombinant methods to obtain a high mannose glycoprotein of interest, can include further manipulation of the transformed host cells or organism. Thus, for example, knockout organisms lacking one or more genes encoding a protein used to create certain glycans, such as lacking xylosyl transferase (XylT) or fucosyl transferase (FucT) activity, or knockdown organisms where the activity of such genes is dampened, can be used. Other knockout or knockdown genes involve loss or inactivity of, for example, N-acetylglucosaminyltransferase 1 (GnT1) or other hexosaminidase. Certain knockin organisms also can be used, such as having a host transformed to express elements that prevent movement of glycoproteins from the ER to Golgi or elements expressing high levels of mannosyl transferases.

The glycoproteins of interest also can be synthesized or tailored using known starting materials or reagents of interest, as known in the art. Thus, a base structure, such as, HIV gp120 that is not glycosylated, for example, made in bacteria, can be treated with a suitable enzyme and suitable reagents to incorporate mannosyl residues onto the protein or glycoprotein of interest. Thus, for example, mannosyl transferases can be used to add mannosyl residues to the protein backbone. Alternatively, enzymes can be used to remove certain residues, such as those found on mature, fully processed glycoproteins, such as, a sialidase, a glucosidase, a galactosidase, an N-acetylglucosaminidase, an N-acetylgalactosaminidase, a fucosidase and so on. In that circumstance, using HIV gp120 again as an example, gp120 obtained from a human cell is treated with the various enzymes to remove the particular saccharides from the gp120 leaving a gp120 expressing, for example, only mannose residues in any of a number of configurations. Such a process also may include adding mannose structures to that gp120 once the non-mannose sugars are removed.

Newly developed plant-based systems can be used to produce the high mannose glycoproteins of interest. A recombinant method using a plant cell as host is exemplified herein, with the recombinant glycoprotein product arrested in an immature (other synonyms include intermediate, incompletely processed, precursor, intracellular, proglycoprotein and so on) form to maintain or to obtain the high mannose configuration. Suitable plant host cells include cells of the intact plant, tissues, organs, seeds, callus, cell lines and so on), see, for example, U.S. Pat. Nos. 7,285,416 and 7,268,226). Suitable species include tobacco, corn, rice, duckweed, Arabidopsis, algae and the like.

Other expression systems can be used, such as mammalian cells, such as CHO cells, 293 cells, lymphocytes, other cell lines and so on; fungal cells, such as yeast cells, such as those obtained from Saccharomyces or Picchia; insect cells; and prokaryotic cells, such as E. coli, as known and available in the art, see, for example, U.S. Pat. Nos. 7,291,490 and 7,288,400.

As discussed above, and as known in the art, the high mannose glycoproteins of interest can be obtained and/or isolated from the appropriate cells practicing methods known in the art. For example, the cells can be lysed using, for example, cold shock, and debris removed by centrifugation. The high mannose glycoproteins of interest can be isolated, for example, by affinity chromatography, using, for example, a particular antibody, such as to the protein portion, or to mannose structures; using an aminophenylboronic affinity medium, using a ConA or other lectin matrix and so on. Such glycoprotein isolation kits are commercially available (Sigma, St. Louis, Mo.).

Once isolated, the high mannose glycoproteins of interest can be packaged and stored as known in the art. Thus, the glycoprotein can be suspended in a suitable salt solution or buffer, and then can be stored, generally under reduced temperature conditions, such as in a refrigerator or associated freezer. If lower temperature conditions for storage are warranted or desired, the solution may be configured to contain a cryoprotectant, such as glycerol.

The high mannose glycoproteins of interest can be administered to a host animal to generate antibodies thereto, practicing methods known in the art, using reagents known in the art. Thus, a host animal or cells can be exposed to the high mannose glycoprotein. The exposure can be repeated, as known in the art. The exposure can include an adjuvant or other immunostimulant, as known in the art. After a suitable stimulation regimen, serum or medium is collected and presence of antibody to high mannose epitopes is determined practicing methods known in the art, such as Western blot, immunodiffusion, ELISA and so on, using the cognate high mannose glycoprotein as antigen.

The resulting antibody or the cognate antigen either can be used in a diagnostic assay to detect the other of the binding pair, in a configuration as known in the art, such as an ELISA, used in an immunofluorescence assay, used for an in vivo scan with a γcamera and so on. Thus, high mannose glycoprotein or antibody thereto can be labeled using materials and methods as known in the art, and used as a reagent as known in the art, or configured as a design choice. Suitable detectable labels include particles, enzymes, dyes, radionuclides and so on. Thus, the wells of a microtiter plate can be coated with a high mannose glycoprotein of interest using methods known in the art. Then samples suspected of containing an antibody thereto are applied to the wells coated with the high mannose glycoprotein of interest. Presence of bound antibody then is detected with a labeled reporter, as known in the art.

The high mannose glycoprotein of interest also can be formulated as a administrable pharmaceutical preparation as known in the art, see Remington Science and Practice of Pharmacy or Remington's Pharmaceutical Sciences, for example. Thus, a suitable amount of a glycoprotein of interest is mixed with a suitable liquid carrier, such as a sterile buffer or liquid culture medium. The immunogen is suspended or dissolved therein, and the liquid formulation can include other inert ingredients, such as buffers, stabilizers, antimicrobials, flavorants, odorants, colorants and the like, an adjuvant optionally also can be used. The liquid formulation is administered as known in the art, such as IP, IM, IV, SC, in the foot pad, intranasally, vaginally, orally, rectally and so on. As known in the art, multiple administrations of the glycoprotein of interest can be used to enhance the immune response.

The formulation can be prepared in a dried of desiccated form for reconstitution prior to use with a suitable liquid diluent, such as sterile water, to enhance shelf life. The dried form can be made as known in the art, such as, lyophilization.

The glycoprotein also can be administered as a part of or as a product of the host organism as well. Thus, the vaccine can be configured to be present in a tissue, organ or product of a host. Hence, an animal transgenic to express a high mannose glycoprotein of interest can contain said immunogen in tissues thereof, or said immunogen can be expressed in a product therefrom, such as milk. Ingestion of the animal or product thereof, can provide the high mannose glycoprotein of interest to the host ingesting same. Similarly, a plant transgenic to express a high mannose glycoprotein of interest can be ingested to obtain exposure to a high mannose glycoprotein of interest.

Alternative means of administration as known in the art can be used, such as in a depot, such as a suppository or adjuvant, or in a mechanical device for regulated release of agent, in an oral formulation and so on. In those circumstances, the high mannose glycoprotein of interest is formulated accordingly, such as mixed with a lipid meltable at body temperature or a water soluble compound, such as polyethylene glycol (PEG), or glycerin, or with a liquid lipid to form an emulsion with a slow rate of dissolution; or is formulated into a form acceptable for oral administration, such as a syrup, elixir, tablet, capsule and so on. For the solid oral formulations, a high mannose glycoprotein of interest is admixed with various carriers, excipients and diluents, such as celluloses, binders, lubricants and the like, and can be manipulated to form a mixture, such as by dry or wet granulation methods, or spray mixing means, as known in the art. The mixture can be compressed into tablets, or the granulation can be sized to form particles, which can be coated, for inclusion into dissolvable packages, such as a capsule, made from, for example, gelatin. The particles or tablets can be coated, as known in the art, to provide other beneficial properties, such as to mask taste, for delayed release and so on.

A vaccine is used to generate an immunoprotective response. The dosage is derived, extrapolated and/or determined from preclinical and clinical studies, as known in the art. Multiple doses can be administered as known in the art, and as needed to ensure a prolonged prophylactic state. The successful endpoint of the utility of a vaccine for the purpose of this invention is the resulting presence of an induced serum antibody, or antibody made by the host in any tissue or organ, that binds the glycoprotein immunogen of interest, and perhaps preferably a high mannose epitope. In some embodiments, the induced antibody in some way, neutralizes and/or eliminates the pathogen carrying the cognate high mannose glycoprotein of interest. For the purposes of the instant invention, observing immunoprotection of at least thirty days is evidence of efficacy of a vaccine of interest. The time of immunoprotection can be at least 45 days, at least 60 days, at least 3 months, at least 4 months, at least 5 months, at least 6 months, at least 1 year, at least 2 years or longer. Preferably the immunoprotection is observed in outbred populations, and to different forms, strains, variants, alleles and the like of a pathogen.

The invention now will be described in the following non-limiting examples.

### EXAMPLES

The HIV gp145 ΔCFI plasmid (37) has been expressed in plants. This cDNA was derived from the gp160 env gene of HIV-1 HXB2 with the amino acids 275-361 being replaced with the CCR5-tropic HIV-1 Bal sequence (which includes the V3 loop). The construction of gp145 ΔCFI involved a stop codon at position 704 and a series of internal deletions at 503-537 and 593-619 which deleted the (i) cleavage site (to prevent proteolytic processing of the env and stabilizing the protein), (ii) the fusion peptide domain (to reduce toxicity and enhance stability) and (iii) the sequence between the heptad repeats (to stabilize the formation of trimers).

Agrobacteria were transformed with a suitable vector, containing the gp145 ΔCFI insert, as well as the C terminal KDEL sequence for ER targeting and retention, by electroporation. For transient expression, recombinant Agrobacteria were vacuum-infiltrated into tobacco leaves, for example. After infiltration, leaves were incubated adaxial side down on wetted paper in sealed trays at 23° C. with a 16 h photoperiod. After 60 h, leaves were frozen in liquid nitrogen and stored at 80° C. until analyzed.

For transient expression in tobacco suspension cultures, cells were co-cultivated with recombinant Agrobacteria in Petri dishes and harvested after two to three days. After harvesting, the cells were frozen and stored until further processed and analyzed. Suspension cultures, such as the BY2 tobacco cell line, can be used for biomass production.

Transgenic tobacco plants were generated using the leaf-disc transformation method. Transformed cells were selected on kanamycin (20 mg/ml) and regenerated plants were screened for accumulation of gp145 by virtue of KDEL retention in the ER. The transgenic T1 lines were analyzed and again the plants showing highest accumulation of gp145 ΔCFI were selected and kept for obtaining seeds.

For the extraction of transiently expressed recombinant proteins, infiltrated leaves were ground in liquid nitrogen to a fine powder with a mortar and pestle (other extraction methods are also suitable). Soluble protein was extracted with extraction buffer (PBS pH 7.4, 1 M NaCl, protease inhibitor cocktail) and cell debris was removed by two rounds of centrifugation. A Ni²⁺—NTA (nitrolotriacetic acid) column was equilibrated with extraction buffer (binding buffer), and BY2 supernatant (SN) was applied to the column at a constant flow rate. After sample application, the column was washed with binding buffer. Nonspecifically bound proteins were removed with binding buffer containing 25 mM imidazole. His6-tagged gp145 was eluted using buffer containing 250 mM imidazole. The samples containing gp145ΔCFI were further concentrated using a microspin column with 50 kDa MWCO resulting in a significant (˜30x) concentration gp145.

A comparison of a Coomassie stained PAGE gel and the corresponding western blot detected, with HIV gp120 mAbs, 2F5 and 2G12, large amounts of gp145ΔCFI.

Dot blots were performed to investigate the reactivity profile of plant-expressed gp145 compared with CHO produced HIV gp120. All antibodies bound with a similar reactivity pattern to the two antigens.

Based on the dot blot data, the reactivity of plant expressed gp145 envelope with neutralizing mAbs was further analyzed with BIAcore. Antibodies were first captured via immobilized protein A and the sample passed over the sensor surface. Clear binding of the ER-retained gp145 was observed with 2G12, 2F5, IgG1b12 and A32 anti-gp120. Extracts from control wild type tobacco leaves gave no increase in signal and no signal was observed using the inactive 2G12 mutant (one aa replacement in the light chain). While 2F5 binds better to plant gp145 in terms of the on rate, the dissociation rate appeared to be slower with the 2G12 than with any other antibody. To further analyze whether the increased binding of the plant high mannose 2G12 epitope was significant, the BIAcore assay was performed comparing the high mannose form of the plant derived env versus the secreted form of a CHO-derived HIV gp120 env. Plant derived gp145ΔCFI binds very tightly (slower dissociation rate) to the captured 2G12 mAb than to the secreted form, suggesting important structural differences in the env molecules. Two possible explanations are: (i) differences in the high mannose 2G12 epitope on plant derived gp145 versus CHO derived gp120 and (ii) differences in the oligomerisation of the two molecules.

A crude estimation of BY2 gp145 content/processed volume was calculated as follows: processed supernatant (SN) volume was 140 ml with a protein content of ˜150 ng in 15 μl which reduces to 10 ng/μl. The total volume was 7 ml. Thus, the recovery was 70 μg and the yield was 0.5 μg/ml.

Neutralization of primary SHIV 89.6 isolate is measured in MT-2 cells by neutral red to quantify cells that survive virus-induced cytopathic effects. Briefly, 500 TCID₅₀ of virus are incubated with multiple dilutions of samples in a total volume of 150 μl for 1 hr at 37° C. in triplicate in 96-well V-bottom culture plates. Cells are added (5×10⁴ cells/100 μl per well) and the incubation is continued until most but not all of the cells in virus control wells (cells plus virus but no serum sample) are involved in syncytium formation (usually 4-6 days). Neutralizing antibody titers are defined as the serum dilution (before the addition of cells) at which 50% of cells are protected from virus-induced killing. A 50% reduction in cell killing corresponds to 85-90% reduction in viral Gag antigen synthesis in a similar assay. Each set of assays includes suitable positive and negative controls.

All references cited herein, are herein incorporated by reference in entirety.

### LITERATURE CITED

1. Letvin N L, Barouch D H, Montefiori D C. Prospects for vaccine protection against HIV-1 infection and AIDS. Annu Rev Immunol. 2002; 20:73-99.

2. Allen T M, Vogel T U, Fuller D H, Mothe B R, Steffen S, Boyson J E, Shipley T, Fuller J, Hanke T, Sette A, Altman, Moss B, McMichael A J, Watkins D I. Induction of AIDS virus-specific CTL activity in fresh, unstimulated peripheral blood lymphocytes from rhesus macaques vaccinated with a DNA prime/modified vaccinia virus Ankara boost regimen. J Immunol. 2000; 164:4968-78.

3. Seth A, Ourmanov I, Schmitz J E, Kuroda M J, Lifton M A, Nickerson C E, Wyatt L, Carroll M, Moss B, Venzon D, Letvin N L, Hirsch V M. Immunization with a modified vaccinia virus expressing simian immunodeficiency virus (SIV) Gag-Pol primes for an anamnestic Gag-specific cytotoxic T-lymphocyte response and is associated with reduction of viremia after SIV challenge. J Virol. 2000, 74:2502-9.

4. Barouch D H, Santra S, Kuroda M J, Schmitz J E, Plishka R, Buckler-White A, Gaitan A E, Zin R, Nam J H, Wyatt LS, Lifton M A, Nickerson C E, Moss B, Montefiori D C, Hirsch V M, Letvin N L. Reduction of simian-human immunodeficiency virus 89.6P viremia in rhesus monkeys by recombinant modified vaccinia virus Ankara vaccination. J Virol. 2001, 75:5151-8

5. Shiver J W, Fu T M, Chen L, Casimiro D R, Davies M E, Evans R K, Zhang Z Q, Simon A I et al. Replication-incompetent adenoviral vaccine vector elicits effective anti-immunodeficiency-virus immunity. Nature. 2002, 415:331-5.

6. Pal R, Venzon D, Letvin N L, Santra S, Montefiori D C, Miller N R, Tryniszewska E, Lewis M G, et al. ALVAC-SIV-gag-pol-env-based vaccination and macaque major histocompatibility complex class I (A*01) delay simian immunodeficiency virus SIVmac-induced immunodeficiency. J Virol. 2002, 76:292.

7. Klein M R, van Baalen C A, Holwerda A M, Kerkhof Garde S R, Bende R J, Keet I F, Eeftinck-Schattenkerk J K et al. Kinetics of Gag-specific cytotoxic T lymphocyte responses during the clinical course of HIV-1 infection: a longitudinal analysis of rapid progressors and long-term asymptomatics. J Exp Med. 1995, 18:1365-72.

8. McMichael A J, Ogg G, Wilson J, Callan M, Hambleton S, Appay V, Kelleher T, Rowland-Jones S. Memory CD8⁺T cells in HIV infection. Philos Trans R Soc Lond B BioI Sci. 2000; 355:363-7.

9. Barouch D H, Santra S, Schmitz J E, Kuroda MJ , Fu T M, Wagner W, Bilska M, Craiu A, Zheng X X, Krivulka G R, Beaudry K, Lifton M A, Nickerson C E, Trigona W L, Punt K, Freed D C, Guan L, Dubey S, Casimiro D, Simon A, Davies M E, ChaStain M, Strom T B, Gelman R S, Montefiori D C, Lewis M G. Control of viremia and prevention of clinical AIDS in rhesus monkeys by cytokine-augmented DNA vaccination. Science. 2000; 290:486-92.

10. Schmitz J E, Kuroda M J, Santra S, Sasseville V G, Simon M A, Lifton M A, Racz P, Tenner-Racz K, Dalesandro M, Scallon B J, Ghrayeb J, Forman M A, Montefiori D C, Rieber E P, Letvin N L, Reimann K A. Control of viremia in simian immunodeficiency virus infection by CD8⁺lymphocytes. Science. 1999; 283:857-60.

11. Jin X, Bauer D E, Tuttleton S E, Lewin S, Gettie A, Blanchard J, Irwin C E, Safrit J T, Mittler J, Weinberger L, Kostrikis L G, Zhang L, Perelson A S, Ho D D. Dramatic rise in plasma viremia after CD8(+) T cell depletion in simian immunodeficiency virus-infected macaques. J Exp Med. 1999; 189:991-8.

12. Kuroda M J, Schmitz J E, Charini W A, Nickerson C E, Lifton M A, Lord C I, Forman M A, Lewin N L. Emergence of CTL coincides with clearance of virus during primary simian immunodeficiency virus infection in rhesus monkeys. J Immunol. 1999; 162(9):5127-33.

13. Ogg G S, Jin X, Bonhoeffer S, Dumbar P R, Quantitation of HIV-1-specific cytotoxic T lymphocytes and plasma load of viral RNA. Science. 1998; 279:2103-6.

14. Koup R A, Safrit J T, Cao Y et al. Temporal association of cellular immune responses with the initial control of viremia in primary human immunodeficiency virus type 1 syndrome. J Virol. 1994; 68:4650-5

15. O'Connor D H, Allen T M, Vogel T U, Jing P, DeSouza I F, Dodds E, Dunphy E J, Melsaether C, Mothe B, Yamamoto H, Horton H, Wilson N, Hughes A L, Watkins D I. Acute phase cytotoxic T lymphocyte escape is a hallmark of simian immunodeficiency virus infection. Nat. Med. 2002; 8:493-9.

16. Vogel T V, Friedrich T C, O′Connor D H, Rehrauer W, Dodds E I, Hickman H et al. Escape in one of two cytotoxic T-lymphocyte epitopes bound by a high-frequency major histocompatibility complex class 1 molecule, Mamu-A*02: a paradigm for virus evolution and persistence? J Virol. 2002, 76:11623-36.

17. Sanders R W, Venturi M, Schiffner L, Kalyanaraman R, Katinger H, Lloyd K O, Kwong P D, Moore J P. The mannose-dependent epitope for neutralizing antibody 2G12 on human immunodeficiency virus type 1 glycoprotein gp120. J Virol. 2002, 76:7293-305.

18. Parker C E, Deterding L J, Hager-Braun C, Binley J M, Schulke N, Katinger H, Moore J P, Tomer K B. Fine definition of the epitope on the gp41 glycoprotein of human immuno-deficiency virus type 1 for the neutralizing monoclonal antibody 2F5. J Virol. 2001, 75:10906-11.

19. Mascola J R, Stiegler G, VanCott T C, Katinger H, Carpenter C B, Hanson C E, Beary H, Hayes D, Frankel S S, Birx D L, Lewis M G. Protection of macaques against vaginal transmission of a pathogenic HIV-1/SIV chimeric virus by passive infusion of neutralizing antibodies. Nat. Med. 2000; 6:207-10.

20. Baba T W, Liska V, Hofmann-Lehmann R, Vlasak J, Xu W, Ayehunie S, Cavacini L A, Posner M R, Katinger H, Stiegler G, Bernacky B J, Rizvi T A, Schmidt R, Hill L R, Keeling M E, Lu Y, Wright J E, Chou T C, Ruprecht R M. Human neutralizing monoclonal antibodies of the IgG 1 subtype protect against mucosal simian-human immunodeficiency virus infection. Nat. Med. 2000, 6:200-6.

21. Scanlan C N, Pantophlet R, Wormald M R, Ollmann Saphire E, Stanfield R, Wilson I A, Katinger H, Dwek R A, Rudd P M, Burton D R. The broadly neutralizing anti-human immunodeficiency virus type 1 antibody 2G12 recognizes a cluster of α1→2mannose residues on the outer face of gp120. J Virol. 2002, 76:7306-21.

22. Sanders R W, Venturi M, Schiffner L, Kalyanaraman R, Katinger H, Lloyd K O, Kwong P D, Moore J P. The mannose-dependent epitope for neutralizing antibody 2G12 on human immunodeficiency virus type 1 glycoprotein gp120. J Virol. 2002, 76:7293-305.

23. Li M, Gao F, Mascola J R, Stamatatos L, Polonis V R, Koutsoukos M, Voss G, Goepfert P, Gilbert P, Greene K M, Bilska M, Kothe D L, Salazar-Gonzalez J F, Wei X, Decker J M, Hahn B H, Montefiori D C. Human immunodeficiency virus type 1 env clones from acute and early subtype B infections for standardized assessments of vaccine-elicited neutralizing antibodies. J Virol. 2005, 79:10108-25.

24. Dacheux L, Moreau A, Ataman-Onal Y, Biron F, Verrier B, Barin F. Evolutionary dynamics of the glycan shield of the human immunodeficiency virus envelope during natural infection and implications for exposure of the 2G12 epitope. J Virol. 2004, 78:12625-37.

25. Stoger E, Sack M, Nicholson L, Fischer R, Christou P. Recent progress in plantibody technology. Curr Pharm Des. 2005;11:2439-57.

26. Means R E, Matthews T, Hoxie J A, Malim M H, Kodama T, Desrosiers RC. Ability of the V3 loop of simian immunodeficiency virus to serve as a target for antibody-mediated neutralization correlation of neutralization sensitivity, growth in macrophages, and decreased dependence on CD4. J. Virol. 2001, 75:3903-15.

27. Burton D R, Desrosiers R C, Doms R W, Koff W C, Kwong P D, Moore J P, Nabel G J, Sodroski J, Wilson I A, Wyatt R T. HIV vaccine design and the neutralizing antibody problem. Nat. Immunol. 2004, 5:233-6.

28. Cutalo J M, Deterding L J, Tomer K B. Characterization of glycopeptides from HIV-I(SF2) gp120 by liquid chromatography mass spectrometry. J Am Soc Mass Spectrom. 2004, 15:1545-55.

29. Haynes B F, Fleming J, St Clair E W, Katinger H, Stiegler G, Kunert R, Robinson J, Scearce R M, Plonk K, Staats H F, Ortel T L, Liao H X, Alam S M. Cardiolipin polyspecific autoreactivity in two broadly neutralizing HIV-1 antibodies. Science. 2005, 308:1906-8.

29. Meffre E, Schaefer A, Wardemann H, Wilson P, Davis E, Nussenzweig M C. Surrogate light chain expressing human peripheral B cells produce self-reactive antibodies. J Exp Med. 2004, 199:145-50.

30. Warier S V, Pinter A, Honnen W J, Girard M, Muchmore E, Tilley S A. A novel, glycan-dependent epitope in the V2 domain of human immunodeficiency virus type 1 gp120 is recognized by a highly potent, neutralizing chimpanzee monoclonal antibody. J Virol. 1994, 68:4636-42.

31. Fischer R, Emans N. Molecular farming of pharmaceutical proteins. Transgenic Res. 2000, 9:279.

32. Fischer R, Hoffmann K, Schillberg S, Emans N. Antibody production by molecular farming in plants. J Biol Regul Homeost Agents. 2000, 14:83-92.

33. Koo M, Bendahmane M, Lettieri G A, Paoletti A D, Lane T E, Fitchen J H, Buchmeier M J, Beachy R N. Protective immunity against murine hepatitis virus (MHV) induced by intranasal or subcutaneous administration of hybrids of tobacco mosaic virus that carries an MHV epitope. Proc. Natl. Acad. Sci. U.S.A. 1999; 96:7774-9.

34. Marusic C, Rizza P, Lattanzi L, Mancini C, Spada M, Belardelli F, Benvenuto E, Capone I. Chimeric plant virus particles as immunogens for inducing murine and human immune responses against human immunodeficiency virus type 1. J Virol. 2001, 75:8434-9.

35. Yusibov V, Modelska A, Steplewski K, Agadjanyan M, Weiner D, Hooper D C, Koprowski H. Antigens produced in plants by infection with chimeric plant viruses immunize against rabies virus and HIV-1. Proc Natl Acad Sci USA. 1997; 94:5784-8.

36. McLain L, Porta C, Lomonossoff G P, Durrani Z, Dimmock N J. Human immunodeficiency virus type 1-neutralizing antibodies raised to a glycoprotein 41 peptide expressed on the surface of a plant virus. AIDS Res Hum Retroviruses. 1995; 11:327-34.

37. Chakrabarti B K, Kong W P, Wu B Y, Yang Z Y, Friborg J, Ling X, King S R, Montefiori D C, Nabel G J. Modifications of the human immunodeficiency virus envelope glycoprotein enhance immunogenicity for genetic immunization. J Virol. 2002, 76:5357-68.

38. Schagger H, Cramer W A, von Jagow G. Analysis of molecular masses and oligomeric states of protein complexes by blue native electrophoresis and isolation of membrane protein complexes by two-dimensional native electrophoresis. Anal Biochem. 1994, 217:220-30.

It should be understood that various changes and modifications to the presently preferred embodiments described herein will be apparent to those skilled in the art. Such changes and modifications can be made without departing from the spirit and scope of the present subject matter and without diminishing its intended advantages.

1. A composition comprising an isolated recombinant glycoprotein comprising an immunogenic high mannose epitope and a pharmaceutically acceptable carrier.

2. The composition of claim 1, wherein said glycoprotein comprises a viral glycoprotein.

3. The composition of claim 2, wherein said viral glycoprotein is the envelope protein of human immunodeficiency virus.

4. The composition of claim 1, wherein said glycoprotein is obtained from a plant cell.

5. The composition of claim 4, wherein said plant cell comprises a transgenic plant, a tissue, an organ, a seed or a cell line.

6. The composition of claim 1, wherein said glycoprotein is obtained from a mammalian cell.

7. The composition of claim 1, wherein said glycoprotein is obtained from a yeast cell.

8. The composition of claim 1, wherein said glycoprotein is obtained from an insect cell.

9. The composition of claim 1, wherein said glycoprotein is obtained from a prokaryotic cell.

10. The composition of claim 5, wherein said transgenic plant is a knockout or a knockdown.

11. The composition of claim 1, wherein said glycoprotein comprises a KDEL tetrapeptide at the C terminus thereof.

12. The composition of claim 6, wherein said mammalian cell comprises a cell line.

13. The composition of claim 12, wherein said cell line is CHO or a 293 cell line.

14. The composition of claim 1, wherein said glycoprotein comprises a detectable marker.

15. A method of making a pharmaceutically administrable glycoprotein comprising an immunogenic high mannose epitope, comprising:
(a) obtaining a cell transformed to express a polypeptide comprising a KDEL tetrapeptide at the C terminus thereof;
(b) culturing said cell of step (a);
(c) isolating high mannose glycoproteins from said cell of step (b); and
(d) mixing said glycoprotein of step (c) comprising an immunogenic high mannose epitope with a pharmaceutically acceptable carrier, excipient or diluent to yield said administrable glycoprotein.

16. The method of claim 15, wherein said cell comprises a plant cell.

17. The method of claim 15, wherein said cell comprises a mammalian cell.

18. The method of claim 16, where said plant cell comprises a cell line or a plant.

19. The method of claim 17, wherein said mammalian cell comprises a cell line.

20. The method of claim 19, wherein said cell line is CHO.

21. (canceled)
